(19) United States
(12) Patent Application Publication
(10) Pub. No.: US 2018/0085519 A1
(43) Pub. Date: Mar. 29, 2018

(54) PRESSURE MEASURING CATHETER WITH ADENOSINE INFUSION

(71) Applicant: Medtronic Vascular, Inc., Santa Rosa, CA (US)

(72) Inventors: Gerry McCaffrey, Tuam (IE); John Kelly, Galway (IE); Sean Ward, Dublin (IE)

(21) Appl. No.: 15/279,855

(22) Filed: Sep. 29, 2016

(51) Int. Cl. A61M 5/168; A61M 25/00

(52) U.S. Cl. CPC A61M 5/16804; A61M 25/00; A61M 2202/0468; A61M 2025/0002; A61M 25/005

(57) ABSTRACT

An intracoronary catheter measures pressure distal to a lesion (Pd) and has a fluid lumen for administers adenosine to a patient's vasculature. A controller is configured to provide an injection of adenosine, wait for a period of time, measure Pd, and compare Pd to the Pd measured before the injection of the adenosine. If the difference between the pressures is small or below a threshold, the controller will stop further injections of adenosine because Pd has been determined to be at a relatively constant value. If the pressure difference is large or above a threshold, the controller will provide another adenosine injection, wait for a time period and again measure Pd and compare it to the previous Pd. This process will continue until the measured Pd reaches a relatively constant value.

### FIELD OF THE INVENTION

The invention relates to methods and systems for determining a pressure gradient across a lesion of a vessel for calculating a Fractional Flow Reserve.

### BACKGROUND OF THE INVENTION

The severity of a stenosis or lesion in a blood vessel may be assessed by obtaining proximal and distal pressure measurements relative to the given stenosis and using those measurements for calculating a value of the Fractional Flow Reserve (FFR). FFR is defined as the ratio of a first pressure measurement (Pd) taken on the distal side of the lesion and to a second pressure measurement taken on the proximal side of the lesion usually within the aorta (Pa). Conventionally, a sensor is placed on the distal portion of a guidewire or FFR wire to obtain the first pressure measurement Pd, while an external pressure transducer is fluidly connected via tubing to a guide catheter for obtaining the second or aortic (AO) pressure measurement Pa Calculation of the FFR value provides a lesion specific index of the functional severity of the stenosis in order to determine whether the blockage limits blood flow within the vessel to an extent that treatment is needed. An optimal or normal value of FFR in a healthy vessel is 1.00, while values less than about 0.80 are generally deemed significant and in need of an interventional treatment Common interventional treatment options include balloon angioplasty and/or stent implantation.

If an interventional treatment is required, the interventional device, such as a balloon catheter, is tracked over a guide wire to the site of the lesion. Conventional FFR wires generally are not desired by clinicians to be used as guide wires for such interventional devices. If an intervention treatment is required, the clinician generally removes the FFR wire, inserts a conventional guide wire, and tracks the interventional device to the treatment site over the conventional guide wire. Insertion and retraction of different types of guide wires is not a preferred method of practice by clinicians. Accordingly, there remains a need for a microcatheter to obtain pressure measurements suitable for use in calculating an FFR value for a given stenosis, whereby the clinician may use a conventional or preferential guide wire instead of a FFR guide wire.

Blood flow through the coronary arteries is affected by fluctuations in the pressure arising proximally of the lesion, e.g., in the aorta, as well as fluctuations in pressure arising distally of the lesion, e.g., in the microcirculation. Accordingly, it is not possible to accurately assess the severity of a coronary lesion by simply measuring the pressure differential across the lesion because the pressure measurement taken on the distal side of the lesion is not purely a residual of the pressure transmitted from the aortic end of the vessel. As a result, for an effective calculation of FFR within the coronary arteries, it is necessary to reduce the vascular resistance within the vessel. Currently, pharmacological hyperemic agents, such as adenosine, are administered to reduce and stabilize the resistance within the coronary arteries. These vasodilator agents reduce the dramatic fluctuation in resistance to obtain a relatively stable and minimal resistance value. However, adenosine has serious side effects for the patient, such as chest pain, feeling faint, shortness of breath. Therefore, the amount of adenosine administered to the patient needs to be minimized.

### BRIEF SUMMARY OF THE INVENTION

Embodiments hereof relate to a catheter, such as a pressure measurement catheter, including an elongate shaft having a proximal end optionally coupled to a handle or luer fitting and a distal end having a distal opening. The elongate shaft further includes a proximal portion, an intermediate portion, and a distal portion having a distal tip. In the proximal portion of the elongated shaft, a shaft wall may define separate lumens: a guide wire lumen, a fluid lumen, and a pressure sensing wire lumen extending parallel to each other or side-by-side along the proximal portion. The distal portion of the elongate shaft is configured to receive a guide wire in a distal portion of guidewire lumen thereof. The pressure sensing wire lumen may extend to the distal portion of the elongate shaft to be coupled to a pressure sensor disposed at the distal tip for measuring a pressure of a fluid within lumen of vessel that is distal to a lesion or Pd. Adenosine is administered through fluid lumen and provided to a patient's vasculature. A controller is configured to provide an injection of adenosine, wait for a period of time, measure Pd, and compare Pd to the Pd measured before the injection of the adenosine. If the difference between the pressures is small or below a threshold, the controller will stop further injections of adenosine because Pd has been determined to be at a relatively constant or minimum value. If the pressure difference is large or above a threshold, the controller will provide another adenosine injection, wait for a time period and again measure Pd and compare it to the previous Pd This process will continue until the measured Pd reaches a relatively constant value.

Embodiments hereof relate to a catheter, such as a pressure measurement catheter, including an elongate shaft having a proximal end optionally coupled to a handle or luer fitting and a distal end having a distal opening. The elongate shaft further includes a proximal portion, an intermediate portion, and a distal portion having a distal tip. In the proximal portion of the elongated shaft, a shaft wall may define separate lumens: a guide wire lumen, a fluid lumen, and a pressure sensing wire lumen extending parallel to each other or side-by-side along the proximal portion. The distal portion of the elongate shaft is configured to receive a guidewire in a distal portion of guidewire lumen thereof. The pressure sensing wire lumen may extend to the distal portion of the elongate shaft to be coupled to a pressure sensor disposed at the distal tip for measuring a pressure of a fluid within lumen of vessel that is distal to a lesion or Pd. Adenosine is administered through fluid lumen and provided to a patient's vasculature. A controller is configured to provide a continuous administration of adenosine to the patient, during which time, Pd is measured and compared to previous Pd measurements captured during the continuous administration of adenosine to the patient. If the difference between the pressures is small or below a threshold, the controller will stop the administration of adenosine because Pd has been determined to be at a relatively constant or minimum value. If the pressure difference is large or above a threshold, the controller will continue to administer adenosine until Pd reaches a relatively constant value.

Embodiments hereof relate to a catheter, such as a pressure measurement catheter, including an elongate shaft having a proximal end optionally coupled to a handle or luer fitting and a distal end having a distal opening. The elongate shaft further includes a proximal portion, an intermediate portion, and a distal portion having a distal tip. In the proximal portion of the elongated shaft, a shaft wall may define separate lumens: a guide wire lumen, a fluid lumen, and a pressure sensing wire lumen extending parallel to each other or side-by-side along the proximal portion. The distal portion of the elongate shaft is configured to receive a guidewire in a distal portion of guidewire lumen thereof. The pressure sensing wire lumen may extend to the distal portion of the elongate shaft to be coupled to a pressure sensor disposed at the distal tip for measuring a pressure of a fluid within lumen of vessel that is distal to a lesion or Pd. Adenosine is administered through fluid lumen and provided to a patient's vasculature. A controller is configured to provide an injection of adenosine, wait for a period of time, measure Pd, and compare Pd to the Pd measured before the injection of the adenosine. If the difference between the pressures is small or below a threshold, the controller will stop further injections of adenosine because Pd has been determined to be at a relatively constant or minimum value. If the pressure difference is large or above a threshold, the controller will provide another adenosine injection, wait for a time period and again measure Pd and compare it to the previous Pd. This process will continue until the measured Pd levels out or reaches a relatively constant value. Once Pd has reached a relatively constant value and the injections of adenosine have stopped, computing device 40 can take the most recent Pd measurement, as a representative Pd for the FFR calculations. In another embodiment, computing device 40 can take an average of Pd1 and Pd2 as a representative Pd for the FFR calculations. In another embodiment, computing device 40 can select any one of the previous Pd measurements, which are within a defined threshold or target range as a representative Pd for the FFR calculations. In another embodiment, computing device 40 can select any one of the previous Pd measurements, which were measured post adenosine administration as a representative Pd for the FFR calculations. In yet another embodiment, computing device 40 can calculate a mean, median or average Pd based on a series of Pd acquired during the adenosine injections as a representative Pd for the FFR calculations.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a broken view of a system for measuring FFR with a distal portion thereof shown within a vessel including a lesion, the system including a measurement catheter including a pressure sensor and a guidewire, in accordance with an embodiment hereof.

FIG. 2 is a broken view of the catheter of FIG. 1 in partial longitudinal cross-section.

FIG. 3 is a cross-sectional view of the catheter taken along line 3-3 of FIG. 2.

FIG. 4A is a longitudinal cross-section view of the rapid exchange portion of a catheter according to an embodiment of the Invention.

FIG. 4B is a cross-sectional views taken along lines A-A of FIG. 4A.

FIG. 4C is a cross-sectional view taken along lines B-B of FIG. 4A.

FIG. 4D is a cross-sectional view taken along lines A-A of FIG. 4A showing fluid lumen expanded by adenosine flowing therethrough.

FIG. 5 is a schematic of an exemplary adenosine control system integrated within the handle of a catheter according to an embodiment of the Invention.

### DETAILED DESCRIPTION OF THE INVENTION

Specific embodiments of the present invention are now described with reference to the figures, wherein like reference numbers indicate identical or functionally similar elements. While the disclosure refers to illustrative embodiments for particular applications, it should be understood that the disclosure is not limited thereto. Modifications can be made to the embodiments described herein without departing from the scope of the present disclosure. Those skilled in the art with access to this disclosure will recognize additional modifications, applications, and embodiments within the scope of this disclosure and additional fields in which the disclosed examples could be applied. Therefore, the following detailed description is not meant to be limiting. Further, it is understood that the systems and methods described below can be implemented in many different embodiments of hardware. Any actual hardware described is not meant to be limiting. The operation and behavior of the systems and methods presented are described with the understanding that modifications and variations of the embodiments are possible given the level of detail presented.

References to “an example,” “one embodiment,” “an embodiment,” “in certain embodiments,” etc., indicate that the example or embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” and “distally” are positions distant from or in a direction away from the clinician. “Proximal” and “proximally” are positions near or in a direction toward the clinician.

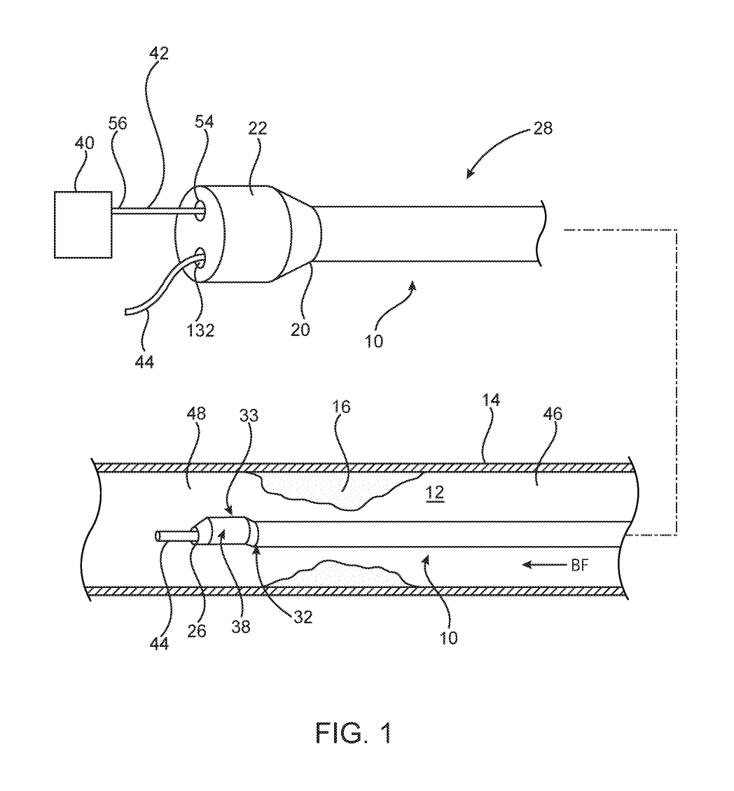

With reference to FIG. 1, a pressure measurement catheter 10 is shown with a proximal portion thereof outside of a patient and a distal portion thereof positioned in situ within a lumen 12 of a patient vessel 14 having a stenosis or lesion 16. In an embodiment hereof, the vessel 14 is a blood vessel such as but not limited to a coronary artery. Lesion 16 is generally representative of any blockage or other structural arrangement that results in a restriction to the flow of fluid through lumen 12 of vessel 14. Lesion 16 may be a result of plaque buildup, including without limitation plaque components such as fibrous, fibro-lipidic (fibro fatty), necrotic core, calcified (dense calcium), blood, fresh thrombus, and mature thrombus. Generally, the composition of lesion will depend on the type of vessel being evaluated. In that regard, it is understood that embodiments hereof are applicable to various types of blockage or other narrowing of a vessel that results in decreased fluid flow.

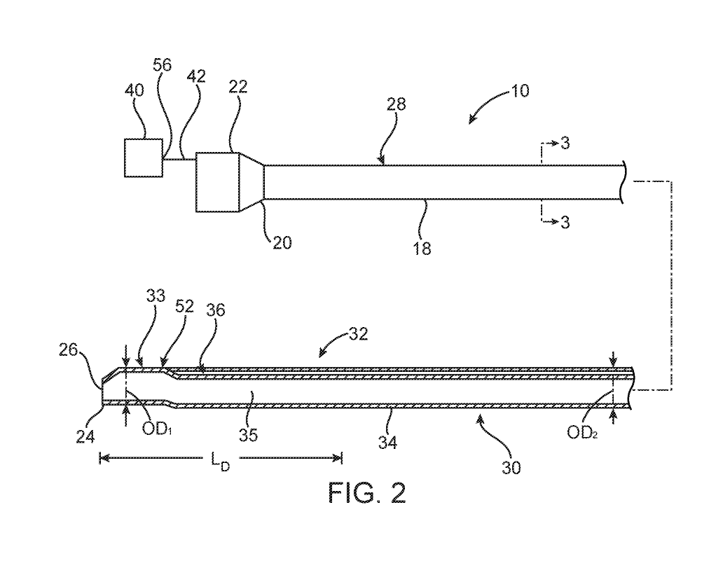

Measurement catheter 10 is shown in FIG. 2 with a distal portion thereof in longitudinal cross-section. Measurement catheter 10 includes an elongate shaft 18 having a proximal end 20 that may be coupled to a handle or luer fitting 22 and a distal end 24 having a distal opening 26. Elongate shaft 18 further includes a proximal portion 28, an intermediate portion 30, and a distal portion 32 having a distal tip 33. Although proximal portion 28, intermediate portion 30, and distal portion 32 of elongate shaft 18 have been described separately, they are described in such a manner for convenience and elongate shaft 18 may be constructed unitarily such that the portions described are part of a unitary shaft. However, different portions of elongate shaft 18 may also be constructed separately and joined together.

In embodiments hereof, elongate shaft 18 or component and/or segments thereof may be formed of polymeric materials, non-exhaustive examples of which include polyethylene terephthalate (PET), polypropylene, polyethylene, polyether block amide copolymer (PEBA), polyimide, fluoropolymers, and/or combinations thereof, either laminated, blended or co-extruded. Optionally, the catheter shaft or some portion thereof may be formed as a composite having a reinforcement material incorporated within a polymeric body in order to enhance strength and/or flexibility. Suitable reinforcement layers include braiding, wire mesh layers, embedded axial wires, embedded helical or circumferential wires, and the like. In one embodiment, for example, at least a proximal portion of elongate shaft 18 may be formed from a reinforced polymeric tube. In other embodiments of an elongate tubular shaft or component in accordance herewith, a proximal segment thereof may be a hypotube of a medical grade stainless steel with outer and inner tubes of a distal segment thereof being formed from any of the polymeric materials listed above.

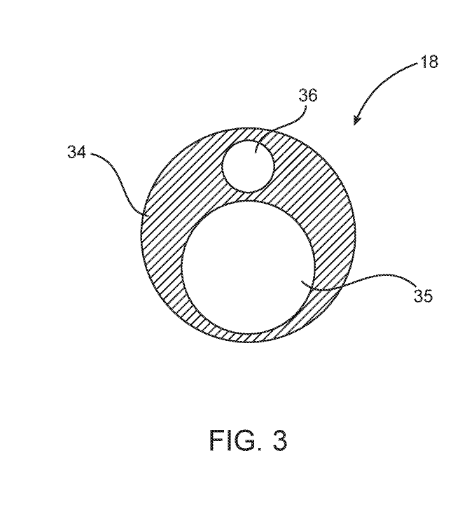

As shown in FIGS. 2-3, elongate shaft 18 has a shaft wall 34 defining a guide wire lumen 35 extending therethrough. Guidewire lumen 35 extends through proximal portion 28, intermediate portion 30, and distal portion 32. In one embodiment, with reference to the cross-sectional view of FIG. 3 (taken along line 3-3 of FIG. 2), in proximal portion 28 of elongated shaft 18, shaft wall 34 defines two separate lumens, guide wire lumen 35 and a fluid lumen 36, extending parallel to each other or side-by-side along proximal portion 28. Although depicted as circular in cross-section, one or more lumen(s) of elongated shaft 18 may have any suitable cross-section including for example circular, elliptical, rectangular or crescent-shaped. As explained in more detail below, fluid lumen 36 may extend to distal portion 32 of elongate shaft 18 in close proximity to a pressure sensor 38 disposed in distal tip 33. Shaft wall 34 has at least one perfusion hole (not shown) disposed adjacent to distal tip 33. The at least one perfusion hole permits a fluid, such as adenosine, to flow from fluid lumen 36 through the at least one perfusion hole in shaft wall 34 and into the patient's vessel. In an optional embodiment, a plurality of perfusion holes are disposed in fluid communication with fluid lumen 36 along the extent of elongated shaft 18. In a further embodiment, the plurality of perfusion holes disposed along the extent of elongated shaft 18 are positioned distally of a rapid exchange port.

Pressure sensor wire or communication wire for pressure sensor 38 are not shown in FIGS. 1-3 for clarity. However, in one embodiment, pressure sensor wire lumen may be eliminated wherein a signal from pressure sensor 38 is sent to a computing device 40 other than via a wire in a dedicated pressure sensor wire lumen, such as, but not limited to, wireless transmission or integration of wire into the wall of elongate shaft 18. In other embodiments of an elongate shaft or tubular component in accordance herewith, a pressure sensor wire lumen may be eliminated wherein the shaft or a portion thereof may be formed by a tubular polymeric inner liner overlaid with a power lead layer and a polymeric outer jacket. In such an embodiment, the power leads for the respective pressure sensor of the inner shaft may be wrapped around the respective shaft for all or at least a portion of the shaft and secured in position by the polymeric outer jacket so as to be embedded within the shaft. In another such embodiment, the power lead for the respective pressure sensor of the inner shaft may be straight for a section or for the entire length of the shaft, and secured in position against the inner liner by the polymeric outer jacket so as to be embedded within the shaft.

Distal portion 32 of elongate shaft 18 is configured to receive a guidewire 44 in a distal portion of guidewire lumen 35 thereof. Further, as shown in FIG. 1, distal portion 32 is sized to extend from a proximal side 46 of lesion 16, through lesion 16, and to a distal side 48 of lesion 16 such that distal tip 33 is disposed on distal side 48 of lesion 16. Accordingly, in an embodiment, distal portion 32 has a length L_{D} in the range of 25-300 mm. However, length L_{D} may be any length suitable such that distal portion 32 may extend from proximal side 46 to distal side 48. Further, because distal portion 32 is configured to extend through lesion 16, the cross-sectional dimension or profile of distal portion 32 is minimized such as to minimize the disruption of blood flow through lesion 16 in order to obtain an accurate FFR measurement.

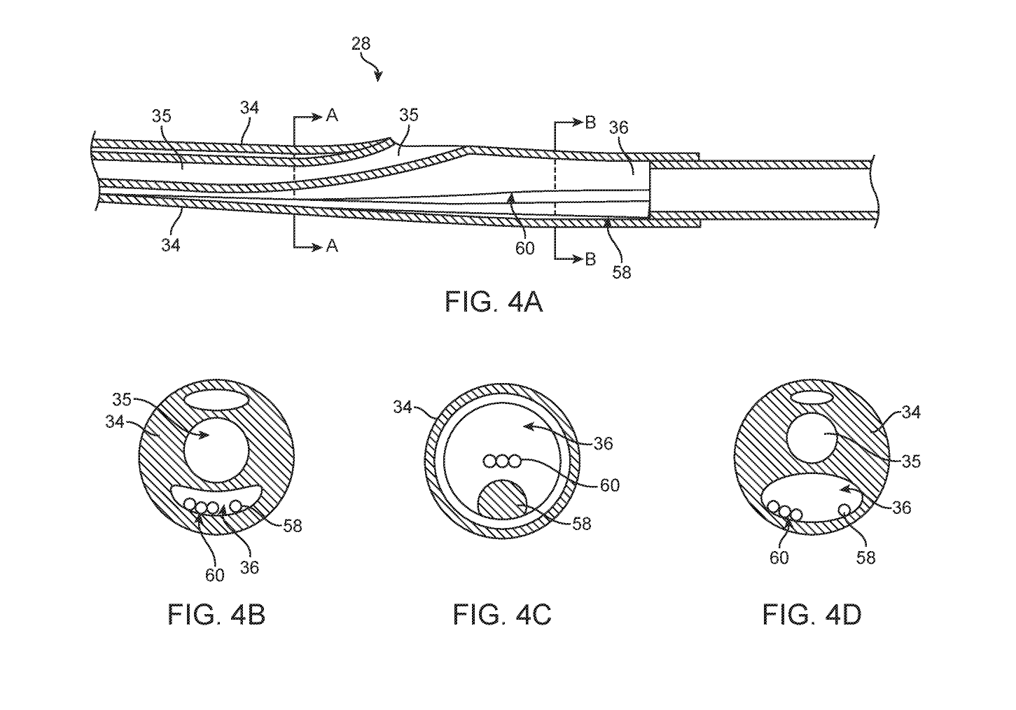

However, instead of the over-the-wire configuration shown in FIGS. 1-3, catheter 10 may have a rapid exchange configuration, as shown in FIGS. 4A-C, wherein guide wire lumen 35 extends through distal portion 32 and intermediate portion 30, and the guidewire 44 exits shaft 18 through a rapid exchange port in proximal portion 28, as would be understood by those skilled in the art. Stiffening wire 58 and tri-filar wire 60 extend along the length of fluid lumen 36. FIG. 4B is a cross-sectional views taken along lines A-A and FIG. 4C is a cross-sectional view taken along lines B-B, wherein in both FIGS. 4B-C, no fluid is disposed within fluid lumen 36.

A method of measuring FFR using measurement catheter 10 will now be described with reference to FIG. 1. As would be understood by those skilled in the art, when measuring FFR a guide catheter (not shown) may be advanced through the vasculature such that the guide catheter is disposed within the aorta with a distal end thereof disposed within the aorta at an ostium of the aorta adjacent the branch vessel 14 within which lesion 16 is located. As shown in FIG. 1, guidewire 44 can be advanced intraluminally through the guide catheter, into vessel 14 within lumen 12 to the site of lesion 16. In the embodiment shown, guidewire 44 is advanced from proximal side 46 of lesion 16 to distal side 48 of lesion 16, which is also consistent with the direction of the blood flow, as indicated by the arrow BF in FIG. 1. In an embodiment, vessel 14 is a coronary artery, but vessel 14 may be other vessels in which it may be desirable to measure pressure, and in particular, to measure FFR.

Thereafter, as shown in FIG. 1, measurement catheter 10 can be tracked or advanced over indwelling guidewire 44 to the target site such that distal end 32 of elongate shaft 18 is positioned distal of lesion 48. As can be seen in FIG. 1, distal tip 33 including pressure sensor 38 can be disposed distally of lesion 16 such that elongate shaft 18 is disposed through lesion 16.

With measurement catheter 10 in place, pressure sensor 38 measures the pressure of blood distal of the lesion within lumen 12. Accordingly, the pressure measured by pressure sensor 33 is the distal pressure measurement, or Pd, used in calculating FFR. A proximal pressure measurement Pa, which is taken in the aorta by an external AO pressure transducer associated with the guide catheter, and a simultaneous pressure measurement Pd taken with pressure sensor 33 of measurement catheter 10 are then obtained to provide the FFR value, i.e., Pd/Pa for the lesion. The proximal pressure measurement Pa and distal pressure measurement Pd can be communicated to computing device 40. Computing device 40, shown schematically in FIGS. 1 and 2, may include such components as a CPU, a display device, an amplification and filtering device, an analog-to-digital converter, and various other components. Computing device 40 may receive the proximal pressure measurement Pa and distal pressure measurement Pd, and may process them to provide a continuous display of FFR measurement.

Vasodilators, such as adenosine, papaverin or the like, dilate the coronary arteries which increase the blood flow, therefore reducing the vascular resistance within the vessel. Vasodilators reduce the dramatic fluctuation in vessel resistance to maintain a relatively stable and minimal resistance value, which is beneficial in obtaining an accurate pressure measurement.

Typically, when performing FFR using a standard guide wire sensor, adenosine is administered either intracoronary at the site, bolus, or intravenously by continuous infusion. Intracoronary injection of the adenosine at the site or by bolus dispenses the adenosine which then travels in the direction of blood flow from proximal side 46 to distal side 48 of lesion 16. Intravenous administration of adenosine is systematic and provides adenosine to all portions of the patient's body. Because adenosine has serious side effects to a patient, such as chest pain, feeling faint, shortness of breath, the amount of adenosine administered to the patient needs to be minimized.

Adenosine travels through fluid lumen 36 and is delivered to distal side 48 of lesion 16. FIG. 4D is an example of FIG. 4B but with a fluid, such as adenosine, disposed within fluid lumen 36. Due to the pressure of adenosine being pumped through fluid lumen 36, fluid lumen 36 is enlarged (as shown in FIG. 4D) due to the wall of fluid lumen 36 expanding in response to the increased pressure. In one embodiment, a pulse of adenosine, as opposed to a bolus of adenosine, is delivered through fluid lumen 36. Thus, fluid lumen 36 would expand and contract to accommodate the pule traveling along the extent of fluid lumen 36. When adenosine is no longer being pumped through fluid lumen 36, the pressure within fluid lumen 36 decreases resulting in fluid lumen 36 deflating or contracting and returning back to its unexpanded shape (as shown in FIG. 4B). The contraction of fluid lumen 36 is advantageous toward minimizing the profile of catheter 10 during the FFR measurement.

In the present Invention, the amount of adenosine is controlled, and only the minimal amount of adenosine will be administered to the patient as is needed for an accurate measurement of pressure. The amount of adenosine delivered to the lesion site via fluid lumen 36 is controlled based on pressure measurements by pressure sensor 38 on distal side 48 of lesion 16. Specifically, an initial injection of adenosine (such as one short burst or pulse of adenosine) is provided distal to lesion 16. After a short waiting period (with no further injections of adenosine), such as about 5-10 seconds, to allow the adenosine to promulgate into the vessel adventitia, a first value of Pd1 is measured. (The waiting time period described herein is exemplary and other time period ranges are within the scope of this Invention since waiting periods can vary from patient to patient.) Then, a second injection of adenosine is provided distal to lesion 16. Once again, a second waiting period of about 5-10 seconds is conducted and a second Pd2 is measured. In an optional embodiment, the second Pd2 is measured before the second injection of adenosine and before the second waiting period. Computing device 40 (or other CPU) compares Pd1 to Pd2 and calculates a difference. If the difference between Pd1 and Pd2 is insignificant or below a threshold percentage, such as 10%, computing device 40 determines that Pd has reached a leveling point or relatively constant value, and therefore further injections of adenosine are not necessary. A visual, audible or other signal can be transmitted by computing device 40 to another device or directly to the operator indicating that further injections of adenosine are no longer needed. The threshold percentage described herein is exemplary and other threshold ranges are within the scope of this Invention.

Once Pd has reached a leveling or constant value due to the influx of adenosine on distal side 48 of lesion, Pd is considered to be approximately at a minimum value. Thus, another reason to stop administering adenosine injections is that an accurate FFR calculation is typically based on minimum values of Pd and any further Pd values would not improve the accuracy of the FFR calculation.

At the point of a leveling Pd (or reaching a constant value for Pd) and with the ceasing of adenosine injections, computing device 40 can derive or calculate a representative Pd for the FFR calculations. For example, the most recent Pd measurement (such as Pd2) can be used as a representative Pd for the FFR calculations. In another embodiment after ceasing adenosine injections, computing device 40 can calculate the mean, median or mode of the Pd1 and Pd2 measurements as a representative Pd for the FFR calculations. In another embodiment after ceasing adenosine injections, computing device 40 can select any one of the previous Pd measurements, orany one of the previous Pd measurements which are within a defined threshold, a targeted range of values or a pre-defined range of values as a representative Pd for the FFR calculations.

In the event, the difference between Pd1 and Pd2 is significant or above a threshold percentage difference, such as 10%, computing device 40 would determine that Pd is still dropping and would therefore administer a third injection of adenosine. After the third injection of adenosine, a third waiting period of about 5-10 seconds is conducted and a third Pd3 is measured. In an optional embodiment, the third Pd3 is measured before the third injection of adenosine and before the third waiting period. Computing device 40 compares Pd2 to Pd3 and calculates a difference. If the difference between Pd2 and Pd3 is insignificant or below a threshold percentage, such as 10%, computing device 40 determines that Pd has reached a minimum value, and therefore no more adenosine needs to be provided. At the point of a leveling Pd (or reaching a constant value for Pd) and with the ceasing of adenosine injections, computing device 40 can derive or calculate a representative Pd for the FFR calculations. For example, computer device 40 can take the most recent Pd measurement (such as Pd3), as a representative Pd for the FFR calculations. In another embodiment after ceasing of adenosine injections, computing device 40 can calculate the mean, median or mode of Pd1, Pd2 and Pd3 as a representative Pd for the FFR calculations. In another embodiment after ceasing of adenosine injections, computing device 40 can select any one of the previous Pd measurements, or any one of the previous Pd measurements which are within a defined threshold, target range of values or a pre-defined range of values as a representative Pd for the FFR calculations. In yet another embodiment, computing device 40 can calculate the mean, median or mode of Pd based on a series of Pd acquired during at least one of the adenosine injections as a representative Pd for the FFR calculations.

If the difference between Pd2 and Pd3 is again significant or above a threshold, computer device 40 can continue to cycle through the above steps until Pd reaches a relatively constant value (or Pd has been determined to reach a minimum value.) Thus, computer device 40 would continue to provide small and controlled injections of adenosine, pausing for time periods after each injection, measuring Pdn after each time period and comparing Pdn with Pd(n−1). In this case, Pdn is the “n^{th}” pressure measurement taken after the n^{th} adenosine injection and n^{th} waiting time period, and Pd(n−1) is the previous pressure measurement taken before the n^{th} adenosine injection and before the n^{th} waiting time period. Alternatively, computing device 40 can compare Pdn with an average of the previous pressure measurements and not be limited to comparing to only the previous Pd(n−1). In another example, computing device 40 can compare Pdn with only previous pressure measurements that have met specific criteria or reached a specific threshold. Qualifying the pressure measurements could reduce errors by preventing the possibility of comparing a faulty pressure reading with an accurate pressure reading. In an optional example, an initial Pdi is measured prior to the first injection of adenosine to provide a baseline pressure measurement at distal side 48 of lesion 16. Thus, Pdi is measured, a first injection of adenosine is provided, a first waiting time period is conducted, Pd1 is then measured and the resulting difference between Pdi and Pd1 is calculated. Based on the difference between Pdi and Pd1, which is compared to a threshold value, further injections of adenosine are either stopped or continued until Pd has reached a constant or minimal value.

In an alternative embodiment, computer device can continually monitor Pd before during or after a single injection or a series of injections of adenosine. Then, once Pd has reached a constant or consistent value, the injection or injections of adenosine can be stopped. As an example, computer device 40 starts the first injection of adenosine at distal side 48 of lesion 16. After a first waiting period, computer device 40 begins either continuous or periodic measurements of Pd. The continuous or periodic Pd measurements represent a first set of a plurality of Pd measurements. Computer device 40 continuously or periodically compares the differences between the plurality of Pd measurements of the first set. For example, within the first set of Pd measurements, the most recent Pdn is compared with the previous measurement Pd(n−1) and calculates the difference between Pdn and Pd(n−1). If the difference between Pdn and Pd(n−1) is below a threshold percentage, such as 10%, computing device 40 determines that Pd has reached a relatively constant (or minimum) value, and would therefore not provide a second injection of adenosine. In addition to comparing the difference between the first set of plurality of Pd measurements, several other methods are possible to determine if Pd has reached a leveling point or relatively constant value. In another embodiment after ceasing adenosine injections, computing device 40 can take the mean, median or mode of the plurality of Pd measurements as a representative Pd from the first set for the FFR calculations. In another embodiment after ceasing adenosine injections, computing device 40 can select any one of the plurality of Pd measurements, or any one of the plurality of Pd measurements which are within a defined threshold, a targeted range of values or a pre-defined range of values as a representative Pd from the first set for the FFR calculations. In the case where the difference between Pdn and Pd(n−1) is above a threshold percentage, such as 10%, computing device 40 would determine that Pd has not yet reached a minimum value, and therefore a second injection of adenosine would be delivered to fluid lumen 36. After a second waiting period, the computer device 40 begins either continuous or periodic measurements of Pd. The continuous or periodic Pd measurements represent a second set of a plurality of Pd measurements. Computer device 40 continuously or periodically compares the difference between the plurality of Pd measurements of the second set. For example, within the second set of Pd measurements, the most recent Pdn is compared with the previous measurement Pd(n−1) and calculates the difference between Pdn and Pd(n−1). The second set of measurements (or a representative Pd from the second set of measurement which can be selected by various methods as described herein) is compared with the first set of measurements (or a representative Pd from the first set of measurements.) If the difference between the first and second set of a plurality of Pd measurements is again significant or above a threshold, computer device 40 can continue to cycle through the above steps until a computed value of Pd reaches a relatively constant value or Pd has been determined to reach a minimum value, at which point adenosine injections would cease. In this alternative embodiment, an initial Pdi can also be measured prior to the injection of adenosine to provide a baseline pressure measurement at distal end 48 of lesion. Thus, Pdi is measured, the first injection of adenosine is delivered to fluid lumen 36, and computer device 40 continually or periodically measures Pd and continuously and periodically compares Pd with Pdi until Pdn has reached a relatively constant (or minimal) value, whereby the flow of adenosine is stopped. In an optional embodiment, computer device 40 continuously compares the differences of the first set of the plurality of pressure measurements to a threshold value while adenosine is being delivered to the fluid lumen. Thus, instead of pausing the adenosine injections to take pressure measurements, computer device 40 will continue to provide adenosine (by way of bolus or pulse delivery) to fluid lumen 36 until the differences between the first set of the plurality of pressure measurements have reached a relatively constant (or minimal) value, or until the differences between the first set of the plurality of pressure measurements is at or near a threshold value.

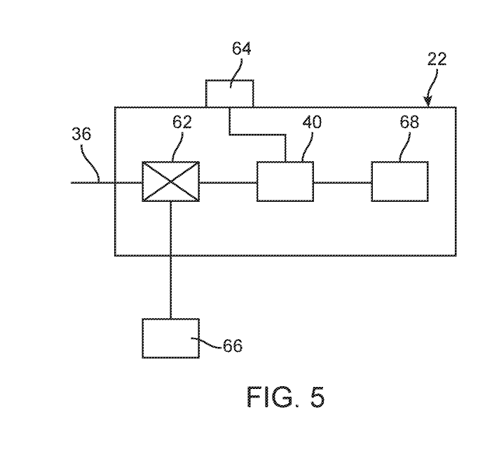

The above described methods help reduce and minimize the amount of adenosine injections into the patient's vasculature. FIG. 5 is one embodiment of an adenosine control system integrated into handle 22 of catheter 10. Computer device 40, CPU or other microcontroller can be integrated into handle 22. Computer device 40 is coupled to valve 62 and a controller 64. Computer device 40 is also coupled to pressure sensor wires or communication wires (not shown in FIG. 5) which in turn are coupled to pressure sensor 38. Valve 62 controls the flow of fluid, such as adenosine, within fluid lumen 36. Adenosine could be supplied, for example, from a power injector 66 in the cath lab or other source. During the pressure measurements, computer device 40 controls the opening and closing of valve 62. If an injection of adenosine is required, computer device 40 opens valve 62 and allows adenosine to flow to the treatment site. Conversely, if adenosine is no longer required, computer device 40 closes valve 62 to stop the flow of adenosine to the treatment site. In a n optional example, controller 64 provides inputs from the operator to computer device 40 in order to provide the operator with manual control over the adenosine injections or FFR measurements. In another optional example, a wireless device 68 is integrated into handle 22 and coupled to computer device 40 in order to provide wireless transmissions between computer device 40 and the cath lab or other external computer or support system.

When the FFR measurement is completed, measurement catheter 10 may then be completely withdrawn from the patient or repositioned in vivo at another lesion and the process repeated. Pressure-sensing guide wires may also be used instead of pressure sending catheters in accordance with the embodiments described herein. Pressure-sensing catheters (or guide wires) in accordance with embodiments hereof may be used for other than providing proximal and distal pressure measurements (Pa, Pd) for calculating an FFR value. For instance, pressure-sensing catheters (or guide wires) in accordance with embodiments hereof may be used to provide an in vivo pressure measurement anywhere along the vasculature, or a particular lesion therein. As well, embodiments hereof may be used to provide in vivo pressure measurements, across a heart valve, venous valve or other valvular locations within the body where it may be deemed useful.

The detailed description is merely exemplary in nature and is not intended to limit the invention or the application and uses of the invention. Although the description of the invention is in the context of treatment of blood vessels such as the coronary arteries, the invention may also be used in any other body passageways where it is deemed useful such as but not limited to peripheral arteries, carotid arteries, renal arteries, and/or venous applications. Furthermore, there is no intention to be bound by any expressed or implied theory presented in the preceding technical field, background, brief summary or the detailed description.

What is claimed is:

1. A catheter comprising:
an elongate shaft having a guide wire lumen and a fluid lumen, the fluid lumen extends to a distal opening at a distal end of the shaft;
a pressure sensor is coupled to the distal end of the elongate shaft;
a control device is coupled to elongate shaft and in fluid communication with the fluid lumen, the control device is configured to control the amount of fluid provided to the fluid lumen, wherein the control device is configured to pause delivery of the fluid while the control device compares the difference between at least two pressure measurements to a threshold value.

2. The catheter of claim 1, wherein the fluid is a vasodilator.

3. The catheter of claim 1, wherein a stiffening wire is disposed within the fluid lumen.

4. The catheter of claim 1, wherein at least one pressure sensor wire is disposed within the fluid lumen.

5. The catheter of claim 1, wherein the fluid is expelled distally of the pressure sensor.

6. The catheter of claim 1, wherein the fluid lumen is radially expandable when fluid is supplied therethrough.

7. The catheter of claim 1, wherein the control device is configured to provide additional fluid to the fluid lumen if the difference of at least two pressure measurements is above the threshold value.

8. The catheter of claim 7, wherein after the additional fluid is provided to the fluid lumen and after at least a third pressure measurement is obtained, the control device is configured to pause delivery of the fluid while the control device compares the difference between a third pressure measurement and at least one of the previous two pressure measurements to the threshold value.

9. The catheter of claim 7, wherein after the additional fluid is provided to the fluid lumen and after at least a third pressure measurement is obtained, the control device is configured to pause delivery of the fluid while the control device compares the difference between an average of at least two of the previous pressure measurements to the threshold value.

10. The catheter of claim 7, wherein after the additional fluid is provided to the fluid lumen and after at least a third pressure measurement is obtained, the control device is configured to pause delivery of the fluid while the control device compares at least one of the previous pressure measurements to the threshold value.

11. The catheter of claim 10, wherein the at least one of the previous pressure measurements are within a pre-defined range of values before being compared to the threshold value.

12. The catheter of catheter of claim 1, wherein the control device is configured to stop the delivery of fluid to the fluid lumen if the difference of the at least two pressure measurements is below the threshold value.

13. A catheter comprising:
an elongate shaft having a guide wire lumen and a fluid lumen, the fluid lumen extends to a distal opening at a distal end of the shaft, wherein the fluid lumen is radially expandable when fluid is supplied therethrough;
a pressure sensor is coupled to the distal end of the elongate shaft;
a control device is coupled to elongate shaft and in fluid communication with the fluid lumen, wherein the control device is configured to control the amount of fluid provided to the fluid lumen based on a first set of a plurality of pressure measurements from the pressure sensor, the control device is further configured to pause delivery of the fluid while the control device continuously compares the differences between the first set of the plurality of pressure measurements to a threshold value.

14. The catheter of claim 13, wherein the fluid is a vasodilator.

15. The catheter of claim 13, wherein a stiffening wire is disposed within the fluid lumen.

16. The catheter of claim 13, wherein at least one pressure sensor wire is disposed within the fluid lumen.

17. The catheter of claim 13, wherein the fluid is expelled distally of the pressure sensor.

18. The catheter of claim 13, wherein the fluid lumen is radially expandable when fluid is supplied therethrough.

19. The catheter of claim 13, wherein the control device is configured to provide additional fluid to the fluid lumen if the difference between the first set of the plurality of pressure measurements is above the threshold value.

20. The catheter of claim 19, wherein after the additional fluid is provided to the fluid lumen and after a second set of a plurality of pressure measurements are obtained, the control device is configured to pause delivery of the fluid while the control device continuously compares the difference between the second set of the plurality of pressure measurements to the threshold value.

21. The catheter of claim 19, wherein after the additional fluid is provided to the fluid lumen and after a second set of a plurality of pressure measurements are obtained, the control device is configured to pause delivery of the fluid while the control device compares the difference between an average of the second set of a plurality of pressure measurements are obtained to the threshold value.

22. The catheter of claim 19, wherein after the additional fluid is provided to the fluid lumen and after a second set of a plurality of pressure measurements are obtained, the control device is configured to pause delivery of the fluid while the control device compares the first and second set of the plurality of measurements to the threshold value.

23. The catheter of claim 13, wherein the first set of the plurality of pressure measurements are within a pre-defined range of values before being compared to the threshold value is.

24. The catheter of catheter of claim 13, wherein the control device is configured to stop the delivery of fluid to the fluid lumen if the difference of the first set of the plurality of pressure measurements is below the threshold value.

25. A catheter comprising:
an elongate shaft having a guide wire lumen and a fluid lumen, the fluid lumen extends to a distal opening at a distal end of the shaft, wherein the fluid lumen is radially expandable when fluid is supplied therethrough;
a pressure sensor is coupled to the distal end of the elongate shaft;
a control device is coupled to elongate shaft and in fluid communication with the fluid lumen, wherein the control device is configured to control the amount of fluid provided to the fluid lumen based on a first set of a plurality of pressure measurements from the pressure sensor, wherein the control device continuously compares the difference of the first set of the plurality of pressure measurements to a threshold value while delivering fluid to the fluid lumen, the control device is further configured to pause delivery of the fluid when the difference between the first set of the plurality of pressure measurements is at or near to the threshold value.
